(19) United States
(12) Patent Application Publication
(10) Pub. No.: US 2008/0108674 A1
(43) Pub. Date: May 8, 2008

(54) CATIONIC ANTISEPTIC AND DYE FORMULATION

(75) Inventors: Angel G. Magallon, El Paso, TX (US); Kyle W. Boone, El Paso, TX (US); James R. Bardwell, El Paso, TX (US); Laura Yanira Hernandez, El Paso, TX (US); Myriam Perez De La Rosa, El Paso, TX (US)

Correspondence Address: SHOOK, HARDY & BACON LLP;INTELLECTUAL PROPERTY DEPARTMENT, 2555 GRAND BLVD, KANSAS CITY, MO, 64108-2613, US

(73) Assignee: ENTURIA, INC., LEAWOOD, KS (US)

(21) Appl. No.: 11/932,304

(22) Filed: Oct. 31, 2007

Related U.S. Application Data
(63) Continuation-in-part of application No. 11/381,045, filed on May 1, 2006.
(63) Continuation-in-part of application No. 11/381,047, filed on May 1, 2006.

(51) Int. Cl. A61K 31/44; A61K 31/155; A61P 31/00

(52) U.S. Cl. 514/358; 514/636; 514/635

(57) ABSTRACT

Aqueous antiseptic solutions and compatible dyes and methods for making and using such solutions are provided. Specifically, in one embodiment, the present invention relates to an antiseptic solution comprising a micellular complex consisting of a cationic excipient and an anionic dye. The antiseptic solution further includes a cationic antiseptic.

### CROSS-REFERENCE TO RELATED APPLICATIONS

This application is a continuation-in-part of U.S. patent application Ser. No. 11/381,045, entitled “Aqueous Antiseptic Solution and Compatible Anionic Dye for Staining Skin”, filed May 1, 2006 and a continuation-in-part of U.S. patent application Ser. No. 11/381,047, entitled “Aqueous Antiseptic Solution and Compatible Cationic Dye for Staining Skin”, filed May 1, 2006.

### STATEMENT REGARDING FEDERALLY SPONSORED RESEARCH OR DEVELOPMENT

Not applicable.

### BACKGROUND

Antisepsis is the destruction or inhibition of microorganisms that exist on living tissue. Antiseptics kill or prevent the growth of the microorganisms. Commonly used antiseptics include iodine, boric acid, and alcohol. Another type of antiseptic used is cationic antiseptics, which are especially effective antiseptics as they exhibit a strong affinity for binding to skin, a high level of antibacterial activity, and prolonged residual effects. It has been found that cationic antiseptics are rapid acting, persistent and superior preoperative skin preparations and kill more bacteria than traditional iodophors or alcohol. Further, cationic antiseptics exhibit rapid activity against both gram-positive and gram-negative bacteria.

However, because aqueous cationic antiseptic solutions are non-colored or clear liquids, it is difficult for the user to see where the liquid has been applied. However, it is important in many situations of using an antiseptic, such as an aqueous cationic antiseptic solution, for an individual to know where the antiseptic has been applied. For example, antiseptics are often applied to a patient's skin just prior to surgery. It is essential that an individual, such as a nurse or surgeon, be able to see where the preoperative liquid has been applied. In such cases, if the preoperative liquid were to be colored such that the liquid would stain a patient's skin when applied, it would be easier for an individual to discern not only that the antiseptic has been applied but also where the liquid has been applied to the patient's body.

Thus, a need exists for an aqueous, non-turbid antiseptic solution whose application is readily visible on the applied surface.

### BRIEF SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

The present invention provides an antiseptic solution comprising a micellular complex consisting of a cationic excipient and an anionic dye; and a cationic antiseptic. In a preferred embodiment, the micellular complex consists of stoichiometric amounts of the excipient and the dye, whereby the total valence charges of the excipient are equivalent to those of the dye. The cationic excipient and the cationic antiseptic may be the same or different compound provided that the solution retains an antiseptic, antimicrobial or germicidal effect. According to one embodiment, the cationic excipient is cetylpyridinium chloride, and in another, the cationic antiseptic is cetylpyridinium chloride or octenidine or salt thereof.

In another aspect of the invention, the solution is substantially clear or translucent. This property is a reflection of the use of the correct stoichiometric ratios of excipient and dye which results in the formation of the micellular complex in solution, rather than precipitate or particulate formation which would otherwise cloud the solution.

The present invention also provides methods of making antiseptic solutions, comprising forming a micellular complex consisting of a cationic excipient and an anionic dye; and combining the complex with a cationic antiseptic, preferably with an aqueous solvent.

The present invention further relates to aqueous antiseptic solutions comprising an aqueous solution of a cationic antiseptic or a salt thereof and a compatible dye in an amount sufficient to stain a patient's skin, by which it is meant that the solution should be sufficiently visible to the healthcare personnel when applied to a patient's skin A dye is compatible in accordance with embodiments of the present invention when an amount sufficient to dye a patient's skin may be dissolved in solution with little or no visible precipitate being formed. Accordingly, a compatible dye used herein will provide an ability to stain a patient's skin when the aqueous antiseptic solution is applied without reducing the efficacy of the cationic antiseptic or salt thereof.

Accordingly, in one aspect, an embodiment of the present invention is directed to an aqueous antiseptic solution comprising an aqueous solution of a cationic antiseptic or a salt thereof, and a cationic dye in an amount sufficient to stain a patient's skin when applied.

Another embodiment of the present invention is directed to an aqueous antiseptic solution comprising an aqueous solution of from about 0.0001, from about 0.01, or from about 0.1% w/v to about 0.5% w/v of octenidine dihydrochloride or a salt thereof, and from about 0.004% w/v to about 0.5% w/v of a cationic dye.

In a further aspect of the invention, an embodiment is directed to a method for preparing an aqueous antiseptic solution having a compatible dye. The method includes adding an amount of cationic dye sufficient to stain a patient's skin to an aqueous solution of a cationic antiseptic or a salt thereof.

Yet another embodiment of the present invention is directed to a method for improving the solubility around a cationic antiseptic or a salt thereof in an aqueous solution. The method includes providing an aqueous solution of a cationic antiseptic or a salt thereof; and providing a cationic dye in an amount that when combined with the aqueous solution of the cationic antiseptic or the salt thereof an antiseptic solution is provided that is capable of staining a patient's skin when applied and in an amount such that the effectiveness of the cationic antiseptic or the salt thereof is not substantially decreased.

In another aspect of the present invention, an embodiment is directed to an aqueous antiseptic solution comprising an aqueous solution of a cationic antiseptic or a salt thereof, an anionic dye in an amount sufficient to stain a patient's skin when applied, and a cationic excipient.

A further embodiment of the present invention is directed to an aqueous antiseptic solution comprising an aqueous solution of from 0.1% w/v to 0.5% w/v of octenidine dihydrochloride or a salt thereof, from 0.07% w/v to 0.30% w/v of an anionic dye, and a cationic excipient, wherein the minimum molar ratio of the cationic excipient to the anionic dye is based on the charge ratio between the cationic excipient and the anionic dye.

In another embodiment, an aspect of the invention is direct to a method for preparing an aqueous antiseptic solution having a compatible dye. The method includes adding to an aqueous solution of a cationic antiseptic or a salt thereof: an amount of anionic dye sufficient to stain a patient's skin, and a cationic excipient.

Still further, an embodiment of the present invention is directed to a method of providing an aqueous antiseptic solution and a compatible dye. The method includes providing an aqueous solution of a cationic antiseptic or a salt thereof. The method also includes providing an anionic dye, wherein the anionic dye is provided in an amount that when combined with the aqueous solution of cationic antiseptic or the salt thereof, such that the antiseptic solution is capable of staining a patient's skin when applied. The method further includes providing a cationic excipient.

Additional aspects of the invention, together with the advantages and novel features appurtenant thereto, will be set forth in part in the description that follows, and in part will become apparent to those skilled in the art upon examination of the following, or may be learned from the practice of the invention. The objects and advantages of the invention may be realized and attained by means, instrumentalities, and combinations particular pointed out in the appended claims.

### DETAILED DESCRIPTION

The present invention is based on the observation that a mixture of a cationic excipient and an anionic dye, in the correct stoichiometric amounts, when combined with a cationic antiseptic, form a clear antiseptic solution. Without being bound by any particular theory regarding a mechanism of action, it is believed that the cationic excipient and the anionic dye form a micellular complex that resists precipitation in an aqueous environment. This clear colored solution is in contrast to prior art compositions that result in turbid solutions.

As used herein, a “cationic excipient” is any molecular entity having at least one positive charge (mono or multivalent) and is capable of forming in an aqueous environment a micellular complex with an anionic dye. Preferably, the cationic excipient is a cationic detergent or surfactant having both a hydrophilic and a lipophilic character that are capable of forming thermodynamically stable micellular solutions. Suitable surfactants are described, for example, in U.S. Pat. No. 5,441,541. For example, the following quaternary ammonium compounds may be used:
[R₁R₂NR₃R₄]⁺X⁻

wherein R₁ is C₈₋₂₂, or more preferably C₁₂₋₂₂ alkyl or alkenyl;

R₂ is C₁₋₆ or more preferably C₁₋₄ alkyl;

R₃ and R₄ are the same or different are selected from the group consisting of C₁₋₆ or more preferably C₁₋₄ alkyl, and —(R₅O)ₙ— wherein R₅ is C₂₋₄, more preferably, C₂₋₃ or C₂ alkylene and n is an integer from 1 to 25, preferably 2 to 20; and

X is a water soluble salt forming anion, such as a halide, e.g., chloride, iodide, or bromide; sulfate, acetate, hydroxide, methosulfate, ethosulfate and the like. Further examples include cetylpyridinium chloride (CPC), hexadecyl trimethyl ammonium bromide, benzethonium chloride, and benzalkonium chloride. In addition, any cationic antiseptic described below having the appropriate properties may be used.

As used herein, a “cationic antiseptic” is any molecular entity having at least one positive charge (mono or multivalent) and an antimicrobial effect. As will be understood by one of skill, a given molecular entity may have properties of both a cationic excipient and an antiseptic, for example, cetylpyridinium chloride.

Examples of cationic antiseptics for use in compositions of the invention include quaternary ammonium compounds, for instance those in which one or two of the substituents on the quaternary nitrogen has between 8 and 20, preferably between 10 and 18 carbon atoms, and is preferably an alkyl group, which may optionally substituted with an amide, ester, oxygen, sulphur, or heterocyclic ring, while the remaining substituents have a lower number of carbon atoms, for instance between 1 and 7, and are preferably alkyl, for instance methyl or ethyl, or benzyl. Examples of such compounds including benzalkonium chloride, dodecyl trimethyl ammonium chloride, dodecyl dimethyl-2-phenoxyethoxyl ammonium bromide, benzyl dimethyl stearyl ammonium chloride, cetyl trimethyl ammonium bromide, cetyl trimethyl ammonium chloride, cetyl trimethyl ammonium tosylate, benzethonium chloride (diisobutyl phenoxyethoxyethyl dimethyl benzyl ammonium chloride) and methyl benzethonium chloride.

Cationic antiseptics may also include pyridinium and isoquinolinium compounds, including hexadecylpyridinium chloride, cetylpyridinium chloride and alkyl isoquinolinium bromide.

Further examples of cationic antiseptics include pyrimidine derivatives such as hexetidine (5-amino-1,3-bis(2-ethylhexyl)-5-methylhexahydropyrimidine); amidine derivatives such as hexamidine isethionate (4,4′-diamidino-α, ω-diphenoxyhexane isethionate); bispyridine derivatives such as octenidine (N,N^{1[}1,10-decanediyldi-1(4H)-pyridinyl-4-ylidene]-bis(1-octanamine dihydrochloride); and biguanides including: monobiguanides such as p-chlorobenzyl biguanide, and N′-(4-chlorobenzyl)-N″-(2,4-dichloro-benzyl) biguanide; and bisbiguanides of the general formula:
A(X)_{Z}NRC(═NH)NHC(═NH)NH(CH₂)ₙNHC(═NH)NHC(═NH)NR′(X′))A′

wherein A and A′ which may be the same or different each represent a phenyl group optionally substituted by (C₁₋₄) alkyl, (C₁₋₄)alkoxy, nitro or halogen, a (C₁₋₁₂) alkyl group, or a (C₄₋₁₂) alicyclic group;

X and X′ which may be the same or different each represent hydrogen or (C₁₋₃)alkylene;

R and R′ which may be the same or different each represent hydrogen, (C₁₋₁₂)alkyl, or aryl(C₁₋₆)alkyl;

z and z′ which may be the same or different are each 0 or 1;

n is an integer from 2 to 12; and the polymethylene chain (CH₂)ₙ may be optionally substituted with oxygen or sulphur or an aromatic (for instance, phenyl or naphthyl) nucleus, and acceptable acid addition salts thereof. Preferred example include chlorhexidine (CHG) and alexidine, and salts thereof such as chlorhexidine gluconate, chlorhexidine digluconate and chlorhexidine acetate; and poly(biguanides) such as polyhexamethylene biguanide hydrochloride.

An exemplary cationic antiseptic that is widely used is chlorhexidine, which has two strongly basic groups. An important feature of its action is the strong binding of chlorhexidine to skin tissue with a subsequent slow release, which maintains an antibacterial action over an extended period. Examples of suitable chlorhexidine salts include gluconate, acetate, chloride, bromide, nitrate, sulphate, carbonate, and phosphanilate.

As used herein, an anionic dye is any molecular entity having at least one negative charge (mono or multivalent) that imparts a color to the aqueous solution, and is capable of forming a complex with the cationic excipient. Anionic dyes that may be used include FD&C dyes, such as, for example, FD&C Blue No. 1 (Brilliant Blue FCF), FD&C Blue No. 2 (Indigo Carmine), FD&C Green No. 3 (Fast Green FCF), FD&C Red No. 3 (Erythrosine), FD&C Red No. 40 (Allura Red), FD&C Yellow No. 5 (Tartrazine), FD&C Yellow No. 6 (Sunset Yellow FCF), and D&C Yellow No. 8 (Fluorescein). One skilled in the art will understand and appreciate that two or more anionic dyes may also be combined and used together in an aqueous antiseptic solution. For instance, an exemplary anionic dye combination is an orange tint that comprises both FD&C Red No. 40 and D&C Yellow No. 8.

Additional suitable excipients, antiseptics and dyes are known in the art (see, for example, WO 04044068; WO 09626724).

As used herein the term “aqueous solution” is used to refer to a solution in which water is the primary dissolving medium or solvent. In other words, the term “aqueous solution” refers to a solution in which water is the solvent in the largest concentration by volume.

The concentration of the cationic antiseptic in the aqueous antiseptic solution may vary within various embodiments of the present invention. Particularly, the concentration of the cationic antiseptic depends on the specific cationic antiseptic used. For example, when using octenidine dihydrochloride or an octenidine salt, the preferred concentration is from about 0.0001, from about 0.01 or from about 0.1% w/v to about 0.5% w/v, and the preferred concentration of chlorhexidine or a chlorhexidine salt is from about 0.5 or from about 2.0% w/v to about 6.0% w/v.

In some embodiments of the present invention, the aqueous antiseptic solution comprises an aqueous solution of an antiseptic, an anionic dye in an amount sufficient to visibly stain a patient's skin when applied, and a cationic excipient in an amount sufficient to substantially prevent the anionic dye from forming a precipitate with the antiseptic. Anionic dyes, including FD&C dyes, can form a precipitate with cationic antiseptics, even at very low concentrations. As such, adding an anionic dye alone to an aqueous cationic antiseptic solution removes a significant fraction of the cationic antiseptic from solution, decreasing the efficacy of the solution.

The precipitate is believed to be the insoluble salt of the cation of the antiseptic and at least one dye anion. The solubility product of the antiseptic-dye complex, assuming one anion, was measured to be less than 10⁻⁹ for all such anionic dyes. However, if the negative charge of an anionic dye is “concealed” from the cationic antiseptic by a cationic excipient, the antiseptic-dye complex will not immediately form. The cationic excipient makes a reversible association with the anionic dye to protect its structure. The antiseptic-dye complex association, however, is an irreversible association when its solubility product is exceeded. While the excipient-dye association may be kinetically favored, the antiseptic-dye complex is thermodynamically favored because the reverse reaction rate is practically zero. However, in preferred embodiments, the addition of a cationic excipient will improve the solubility around the cationic antiseptic by preventing the formation of the antiseptic-dye complex.

It is believed that the interaction between anionic dyes and cationic excipients in aqueous solutions of the present invention comprises a reversible association of the anionic dyes with cationic excipient micelles by ionic interactions. Micellation refers to a process in which submicroscopic molecules aggregate, as a droplet in a colloidal system. The driving force for aggregation is the gain in entropy, according to Boltzmann's principle, of the water molecules formerly associated with the hydrophobic molecules and now associated with other water molecules. Entropy is increased because water has allowable states next to polarized molecules (hydrophilic) than next to non-polarized molecules (hydrophobic). Thus, formation of micellular complexes may be affected by the location of the cationic charge on the cationic excipient. Different structural compounds may provide steric advantages during micellular formation in an aqueous solution. The increase in ionic strength associated with dissolving an anionic dye packs micellular cationic excipient molecules even closer together, increasing the density of positive charge at the surface of the micelle.

In embodiments, the concentration of anionic dye sufficient to stain a patient's skin but otherwise compatible with cationic antiseptics via the addition of a cationic excipient may range from about 0.07% w/v to about 0.30% w/v.

Certain types of cationic excipient may be employed with the scope of the present invention to provide a compatible cationic antiseptic-anionic dye solution. In some embodiments, the cationic excipient comprises a cationic detergent. In some embodiments, a cationic excipient containing quaternary nitrogen may be used. Such cationic excipients may include, for example, cetylpyridinium chloride (CPC), hexadecyl trimethyl ammonium bromide, benzethonium chloride, and benzalkonium chloride. In one embodiment, the cationic excipent, such as cetylpyridinium chloride has a hydrophilic and a lipophilic character that are capable of forming thermodynamically stable micellular solutions. The concentration of cationic excipient is dependent upon the charge ratio between the dye and cationic excipient used to prepare the aqueous antiseptic solution. For example, a cationic excipient having a single positive charge and an anionic dye having two negative charges would result in a molar ratio of cationic excipient to anionic dye of about 2 to 1. In one embodiment, the concentration of a cationic excipient will be from about 0.2% to 2.5% w/v. In one embodiment, the molar ratio of cationic excipient to the anionic dye is about 1.5 to 2.5. The amount of antiseptic may be determined based the desired antimicrobial activity. The amount of anionic dye utilized may be determined by the desired intensity of the dye and color of the solution desired. The amount of cationic excipient may depend on the nature and amount of anionic dye utilized.

In other embodiments of the present invention, the aqueous antiseptic solution comprises an aqueous solution of an antiseptic and a cationic dye in an amount sufficient to visibly stain a patient's skin when applied. Non-limiting examples of such cationic dyes include crystal violet, acriflavine, bismark brown, malachite green, methyl green, Victoria pure blue BO, and azure C. In contrast to anionic dyes, cationic dyes were found to be compatible with aqueous cationic antiseptic solutions without the addition of a cationic excipient. However, some cationic dyes include a chloride ion or other ion that forms a precipitate with the cationic antiseptic as the concentration is increased. For example, the compatibility of cationic dyes having a chloride ion with aqueous cationic antiseptic solutions decreases after the solubility product of the cationic antiseptic and chloride is exceeded at about 0.05% w/v dye. As such, in embodiments, the concentration of cationic dye sufficient to stain a patient's skin but otherwise compatible with a cationic antiseptic may range from about 0.004% w/v to about 0.5% w/v. In preferred embodiments, the concentration of cationic dye is about 0.05% w/v.

Aqueous antiseptic solutions in accordance with some embodiments of the present invention may employ additional components. For example, in some embodiments, the aqueous antiseptic solution may employ a surfactant. Examples of such suitable surfactants include polyvinyl pyrrolidone (PVP) (average molecular weight 10,000) and PVP (average molecular weight 1,300,000). In embodiments, the concentration of surfactant in an aqueous antiseptic solution may generally range from about 0.5% w/v to about 5% w/v. In a preferred embodiment, PVP (average molecular weight 10,000) in added as a surfactant in a concentration of about 1% w/v.

Additionally, in some embodiments, aqueous antiseptic solutions may employ a solubilization aid. Examples of such suitable solubilization aids include polyethylene glycol (PEG) (average molecular weight 200), PEG (average molecular weight 300), PEG (average molecular weight 400), and glycerol. The concentration of a solubilization aid in an aqueous antiseptic solution of embodiments of the present invention may generally range from about 1% v/v to about 49% v/v. In a preferred embodiment, PEG (average molecular weight 200) is added as a solubilization aid in a concentration of about 1% v/v to about 49% v/v.

Additional additives may also be employed within aqueous antiseptic solutions of further embodiments of the present invention, including, for example, small concentrations of alcohol. Such additives would be employed in acceptable manners and amounts established in the art.

In some embodiments, an aqueous antiseptic solution and compatible dye may be provided in conjunction with a liquid applicator. For example, a liquid applicator may be provided that comprises a hollow body defining an internal chamber to receive at least one ampoule formed of a frangible material. In some embodiments, the ampoule(s) contain an aqueous antiseptic solution having a dye therein as described hereinabove. The ampoule(s) may be fractured, and the colored aqueous antiseptic solution may be applied to the desired surface. In other embodiments, the ampoule(s) contain an untinted aqueous cationic antiseptic solution, and the liquid applicator includes a porous element with a compatible dye therein. The porous element is positioned such that upon fracturing the ampoule(s), the untinted aqueous antiseptic solution is passed through the porous element and dye is transferred to the solution, which may then be applied to the desired surface. Examples of such liquid applicators are further described in: U.S. Pat. No. 6,729,786, U.S. Pat. No. 6,991,393, and U.S. Pat. No. 7,241,065; each of which is herein incorporated by reference in its entirety.

The ampoule(s) may be numerous different shapes and sizes depending on the amount of liquid needed to be applied. For example, a liquid applicator may include long cylindrical ampoule(s) or may contain vial-type ampoule(s). Furthermore, more than one ampoule may be received by the body. Preferably, the ampoule(s) are formed of glass, although other materials are entirely within the scope of the present invention. The wall of the ampoules is of a thickness sufficient to contain the desired liquid during transport and storage, yet allow the ampoule to be fractured upon the application of localized pressure.

The body of the liquid applicator may take many forms. The body has an internal chamber that is adapted to receive at least one ampoule. The body may also be shaped to hold multiple ampoules. In one form, the body is shaped to generally conform to the ampoule(s) contained within the body.

The porous element of the present invention also may take many forms. The porous element may be a porous plug and/or a porous pad. In other words, a porous plug may be located within the body of the applicator between the ampoule and an open end of the body. Additionally or alternatively, a porous pad may be located at an open end of the body. In some embodiments, a compatible dye (e.g., a cationic dye or an anionic dye/cationic excipient composition) may be provided in and/or on the porous element. The porous element is positioned such that when the ampoule(s) is fractured, the untinted aqueous antiseptic solution flows through the porous element and dye is transferred to the solution to be applied. The porous element may be made of any porous material that allows liquid to flow through the material. The porous element may be, but is not limited to, a fabric, foam or a felt material. Dye may be saturated throughout the porous element or may be placed only on part of the element.

The ampoule(s) contained within the body of the applicator may be broken by any method known to those skilled in the art. These include, but are not limited to, squeezing the walls of the body inwardly to break the ampoule(s), using a lever or other mechanism to break the ampoule(s), or utilizing projecting wings with tappets.

### EXAMPLES

Embodiments of the present invention will now be further illustrated by the following, non-limiting examples.

### Example 1

The compatibility of an anionic dye alone with an aqueous chlorhexidine gluconate (CHG) solution (i.e., without the addition of a cationic excipient) was tested. A 0.13% w/v anionic dye solution was prepared by dissolving 0.13 g FD&C Yellow 6 in 100 ml of distilled water. A 20% w/v aqueous CHG solution was then added drop wise to the dye solution. After two drops of the aqueous cationic antiseptic solution were added to the dye solution, precipitate was formed, demonstrating the incompatibility of the dye alone with the aqueous cationic antiseptic solution.

### Example 2

To test the compatibility of anionic dyes and cationic excipients, a number of solutions were prepared with different anionic dyes and cationic excipients. The cationic excipients tested included: CPC, hexadecyl trimethyl ammonium bromide, benzethonium chloride, and benzalkonium chloride. The anionic dyes tested included: FD&C Green No. 3 (Fast Green FCF), FD&C Yellow No. 5 (Tartrazine), FD&C Red No. 40 (Allura Red), FD&C Yellow No. 6 (Sunset Yellow FCF), FD&C Blue No. 1 (Brilliant Blue FCF), FD&C Blue No. 2 (Indigo Carmine), D&C Yellow No. 8 (Fluoresceine), and FD&C Red No. 3 (Erythrosine). The chemical structure and chemical category of each of these dyes are presented below.

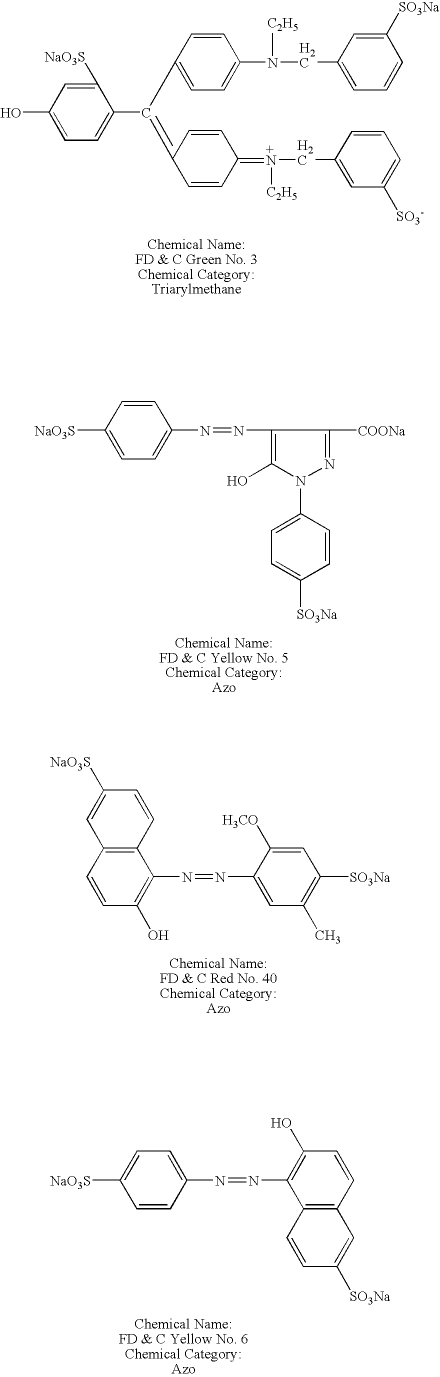

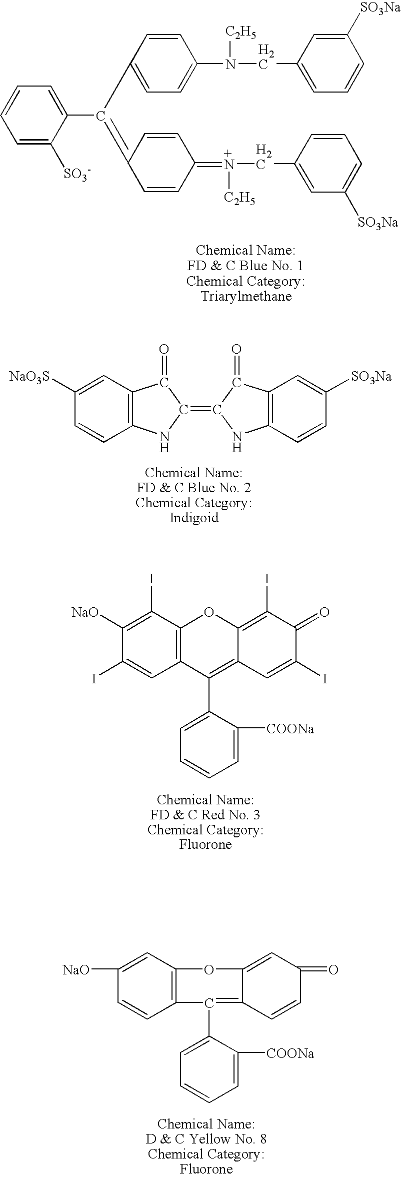

The compatibility of anionic dyes and cationic excipients were tested as follows. First, 0.1 grams of each anionic dye were placed in separate 40-ml beakers. 20 ml of 4 mM excipient solution were added to each 40-ml beaker. Each of the cationic excipients solubilized the anionic dyes.

### Example 3

A titration experiment was designed to determine the appropriate cationic excipient to anionic dye molar ratio. The experiment was performed using CPC as the cationic excipient and FD&C Yellow No. 6 as the dye. The titration was done by placing a known volume of 4 mM CPC solution in a beaker and titrating with a 2% w/v solution of FD&C Yellow No. 6. The solution containing CPC and FD&C Yellow No. 6 was added drop wise to an aqueous 2.0% w/v CHG solution. Results indicated that the minimum molar ratio of CPC to FD&C Yellow No. 6 was approximately 2 to 1. This result represents the charge ratio between the two components.

### Example 4

An aqueous 2.0% w/v CHG solution containing an anionic dye was formulated using a Class A 100-ml volumetric flask. The procedure included dissolving 0.30 grams CPC and 0.13 grams FD&C Yellow No. 6 with 6.0 ml of 50/50% v/v of isopropanol and distilled water. Separately, 1.0 grams of PVP (average molecular weight 10,000) was completely dissolved in 30.0 ml of distilled water. Once dissolved, the PVP solution was incorporated with the dye/excipient solution. Consecutively, 5 ml of PEG (average molecular weight 200) were added. Additionally, 10.6 grams of 20% w/v CHG solution was added. Finally, distilled water was added to the flask until the 100-ml mark was reached.

### Example 5

A tinted aqueous 6.0% w/v CHG solution using an anionic dye and cationic excipient was prepared using a Class A 100-ml volumetric flask. First, 0.30 grams CPC and 0.13 grams FD&C Yellow No. 6 was dissolved in the flask using 6.0 ml of 50/50% v/v of isopropanol and distilled water. Consecutively, 31.8 grams of 20% w/v CHG solution were added. Distilled water was then added to the flask until the 100-ml mark was reached.

### Example 6

In this example, a liquid applicator was prepared containing an untinted aqueous CHG solution in an ampoule and anionic dye/cationic excipient composition contained in a porous element. To prepare the aqueous CHG solution, 1 gram of PVP (average molecular weight 10,000) was dissolved in 30 ml of distilled water. Then, 5 ml of PEG (average molecular weight 200) was added. Additionally, 10.6 grams of 20% w/v aqueous CHG solution was provided and dissolved water was added until the 100-ml mark was reached. The aqueous CHG solution was added to a glass ampoule, which was then sealed and placed inside the hollow body of the liquid applicator.

A porous element having an anionic dye/cationic excipient composition was prepared for the liquid applicator as follows. First, 100 ml of a dye solution was prepared by adding 2.0 grams of FD&C Yellow No. 6 and 4.6 grams of CPC in 100 ml of 50/50% v/v of isopropanol and distilled water. The porous element was dipped in the dye solution for 1 minute and then air-dried for 24 hours. The porous element was then secured to the end of the applicator body.

Upon fracturing of the ampoule, the untinted aqueous CHG solution flows through the porous element containing the anionic dye/cationic excipient composition. Dye and cationic excipient are thereby transferred to the aqueous CHG solution as it flows through the porous element. The resulting colored aqueous CHG solution may be applied to a desired surface, such as a patient's skin, thereby both disinfecting and visibly staining the surface.

### Example 7

To prove that chlorhexidine will not precipitate with cationic ammonium-containing dyes, the following dyes were tested: crystal violet, Victoria pure blue BO, methyl green, malachite green, acriflavine, and bismark brown. The chemical structure and chemical category of each of these dyes are presented below. Crystal violet, malachite green, and Victoria pure blue BO all belong to the same chemical family, triarylmethane. Bismark brown and acriflavine each belong to a different chemical family, azo and acridine, respectively.

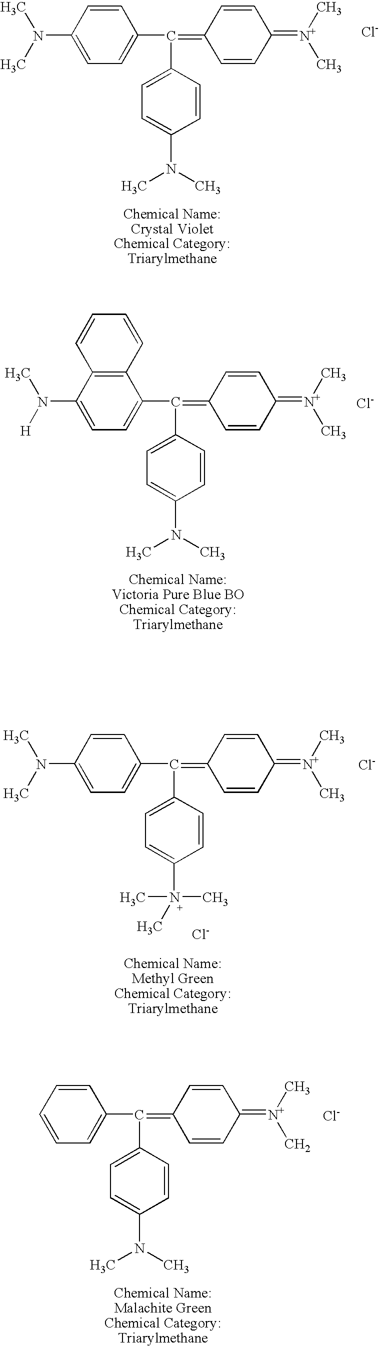

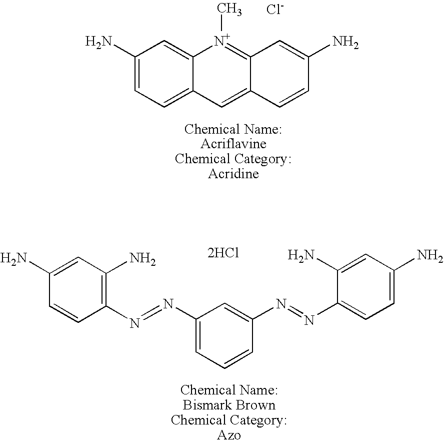

Aqueous solutions of 2.0% w/v CHG and 0.05% w/v dye were prepared for each of the dyes indicated above to test the stability of the solutions. Each aqueous solution was prepared using a Class A 100 ml volumetric flask, in which 0.050 grams of dye were dissolved in 30.0 ml of distilled water. Additionally, 10.6 g of 20% w/v aqueous CHG solution was added to the flask. The solution was then brought up to volume with distilled water. The solutions were stored for three months at room temperatures. No visible precipitate formed in any of the solutions, indicating that the solutions were stable and the dyes were compatible for at least three months at a concentration of 0.05% w/v.

### Example 8

The compatibility of cationic ammonium-containing dyes with aqueous CHG solutions suggested that compatibility may not be limited to ammonium-containing dyes and that any cationic dye may be compatible. Accordingly, an aqueous CHG solution with a cationic dye that does not contain an ammonium group was prepared to test its compatibility. In particular, the dye tested was azure C, which is a cationic dye that belongs to the thiazin family. Its positive charge comes from a tertiary sulfur atom instead of a quaternary nitrogen atom. The chemical structure and chemical category of azure C is presented below.

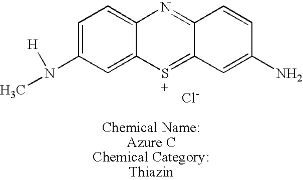

An aqueous solution of 2.0% w/v CHG and 0.05% w/v azure C dye was prepared to test the stability of the solution. The solution was prepared similar to the preparation of the solutions in Example 7. First, 0.050 grams of azure C dye were dissolved in 30.0 ml of distilled water using a Class A 100 ml volumetric flask. Additionally, 10.6 g of 20% w/v aqueous CHG solution was added to the flask. The solution was then brought up to volume with distilled water. The solutions were stored for three months at room temperatures. The solution showed no visible precipitate, indicating that the solution was stable and the dye was compatible for at least three months at a concentration of 0.05% w/v.

### Example 9

An aqueous 6.0% w/v CHG solution containing a cationic dye was prepared using a Class A 100-ml volumetric flask. First, 0.050 grams of crystal violet was dissolved in 30.0 ml of distilled water. Consecutively, 31.8 grams of 20% w/v CHG solution were added. Distilled water was then added to the flask until the 100-ml mark was reached.

### Example 10

In this example, a liquid applicator was prepared containing an untinted aqueous CHG solution in an ampoule and cationic dye contained in a porous element. To prepare the aqueous CHG solution, 10.6 grams of 20% w/v aqueous CHG solution was provided and dissolved water was added until the 100-ml mark was reached. The aqueous CHG solution was added to a glass ampoule, which was then sealed and placed inside the hollow body of the liquid applicator.

A porous element containing a cationic dye was prepared for the liquid applicator as follows. First, 100 ml of a dye solution was prepared by adding 0.3 grams of crystal violet dye in 100 ml of 50/50% v/v of isopropanol and distilled water. The porous element was dipped in the dye solution for 1 minute and then air-dried for 24 hours. The porous element was then secured to the end of the applicator body.

Upon fracturing of the ampoule, the untinted aqueous CHG solution flows through the porous element containing the cationic dye. Dye is thereby transferred to the aqueous CHG solution as it flows through the porous element. The resulting colored aqueous CHG solution may be applied to a desired surface, such as a patient's skin, thereby both disinfecting and visibly staining the surface.

### Example 11

An aqueous solution of 0.1% w/v octenidine dihydrochloride and 0.192% w/v crystal violet was prepared using a Class A 100 ml volumetric flask, in which 0.192 grams of dye were dissolved in 30.0 ml of distilled water. Additionally, 0.1 grams of octenidine dihydrochloride were added to the flask. The solution was then brought up to volume with distilled water. Solutions were also prepared using 0.3% w/v octenidine dihydrochloride with 0.192% w/v crystal violet, and 0.5% w/v octenidine dihydrochloride with 0.192% w/v crystal violet.

The solutions were stored for 24 hours at room temperature. No visible precipitate formed in any of the solutions, indicating that the solutions were stable and the dye was compatible for at least 24 hours at a concentration of 0.192% w/v.

### Example 12

In this example, an aqueous 0.1% w/v octenidine dihydrochloride solution containing a cationic dye was prepared using a Class A 100-ml volumetric flask. First, 0.15 grams of malachite green were dissolved in 30.0 ml of distilled water. Consecutively, 0.1 grams of octenidine dihydrochloride were added. The solution was then brought up to volume with distilled water. Solutions were also prepared using 0.3% w/v octenidine dihydrochloride with 0.150% w/v malachite green, and 0.5% w/v octenidine dihydrochloride with 0.15% w/v malachite green.

The solutions were stored for 24 hours at room temperature. No visible precipitate formed in any of the solutions, indicating that the solutions were stable and the dyes were compatible for at least 24 hours at a concentration of 0.15% w/v.

### Example 13

In this example, an aqueous 0.1% w/v octenidine dihydrochloride solution containing a cationic dye was prepared using a Class A 100-ml volumetric flask. First, 0.15 grams of methyl green were dissolved in 30.0 ml of distilled water. Consecutively, 0.1 grams of octenidine dihydrochloride were added. The solution was then brought up to volume with distilled water. Solutions were also prepared using 0.3% w/v octenidine dihydrochloride with 0.15% w/v methyl green, and 0.5% w/v octenidine dihydrochloride with 0.15% w/v methyl green.

The solutions were stored for 24 hours at room temperature. No visible precipitate formed in any of the solutions, indicating that the solutions were stable and the dyes were compatible for at least 24 hours at a concentration of 0.15% w/v.

### Example 14

A tinted aqueous 0.1% w/v octenidine dihydrochloride solution using an anionic dye and cationic excipient was prepared using a Class A 100-ml volumetric flask. In this example, the anionic dye used was 0.12% w/v orange tint, which consisted of 85% D&C Yellow No. 8 and 15% FD&C Red No. 40. First, 0.12 grams of orange tint, consisting of 0.102 grams D&C Yellow No. 8 and 0.018 grams FD&C Red No. 40, and 0.253 grams CPC were dissolved in the flask using approximately 50 ml of distilled water. Consecutively, 0.1 grams of octenidine dihydrochloride were added. The solution was then brought up to volume with distilled water. For all three solutions, the resulting molar ratio of cationic excipient to dye was 1.84 (where more than one dye is used, the molar ratio is a reflection of the total weight of dyes used).

Solutions were also prepared using 0.3% w/v octenidine dihydrochloride with 0.12% w/v orange tint, and 0.5% w/v octenidine dihydrochloride with 0.12% w/v orange tint. For these solutions, the resulting molar ratio of cationic excipient to dye was also 1.84.

After preparation of the solutions, the solutions had a clear red-orange appearance. The solution was stored for 24 hours at room temperature. During this period, no visible precipitate formed in the solution, indicating that the solutions were stable and the dyes were compatible for at least 24 hours at a concentration of 0.12% w/v orange tint.

### Example 15

In this example, a tinted aqueous 0.1% w/v octenidine dihydrochloride solution using an anionic dye and a cationic excipient was prepared using a Class A 100-ml volumetric flask. First, 1.000 grams CPC and 0.500 grams D&C Yellow No. 8 were dissolved in the flask using 50.0 ml of distilled water. Consecutively, 0.1 grams of octenidine dihydrochloride were added. The solution was then brought up to volume with distilled water. The resulting molar ratio of cationic excipient to dye was 1.91.

The solution was stored for 24 hours at room temperature. During this period, no visible precipitate formed indicating that the solution was stable and the dyes were compatible for at least 24 hours at their original concentrations.

### Example 16

In this example, an aqueous antiseptic solution was prepared using 0.1% w/v octenidine dihydrochloride. In this example, orange tint consisted of 75% D&C Yellow No. 8 and 25% FD&C Red No. 40. The solution was prepared by dissolving 0.12 grams of orange tint, consisting of 0.09 grams of Yellow No. 8 and 0.03 grams of Red No. 40, and 0.246 grams CPC in a Class A 100-ml volumetric flask using 50.0 ml of distilled water. Consecutively, 0.1 grams of octenidine dihydrochloride was added. The solution was then brought up to volume with distilled water. The resulting molar ratio of cationic excipient to anionic dye was 1.79.

Solutions were also prepared using 0.3% w/v octenidine dihydrochloride with 0.12% w/v orange tint, and 0.5% w/v octenidine dihydrochloride with 0.12% w/v orange tint. For these solutions, the resulting molar ratio of cationic excipient to dye was also 1.79.

The resulting solutions were clear and they were stored for 24 hours at room temperature. During this period, no visible precipitate formed in the solution, indicating that the solutions were stable and the dye was compatible for at least 24 hours at the concentration of 0.12% w/v orange tint.

### Example 17

A tinted aqueous 2.0% w/v CHG solution using an anionic dye and cationic excipient was prepared using a Class A 100-ml volumetric flask. In this example, the anionic dye used was 0.25% w/v orange tint, which consisted of 85% D&C Yellow No. 8 and 15% FD&C Red No. 40. First, 0.218 grams CPC and 0.25 grams orange tint, consisting of 0.212 grams of D&C Yellow No. 8 and 0.037 grams of FD&C Red No. 40, were dissolved in the flask using 50.0 ml of distilled water. Consecutively, 10.6 grams of 20% w/v CHG solution were added. The solution was then brought up to volume with distilled water. The resulting solution had a molar ratio of cationic excipient to anionic dye was 1.84.

Solutions were also prepared using 4.0% w/v CHG with 0.12% w/v orange tint, and 6.0% w/v CHG with 0.12% w/v orange tint. For these solutions, the resulting molar ratio of cationic excipient to dye was also 1.84.

After preparation of the 2.0%, 4.0%, and 6.0% w/v CHG solutions were stored for 24 hours at room temperature. During this period, no visible precipitate formed in any of the solutions, indicating that the solutions were stable and the dyes were compatible for at least 24 hours at a concentration of 0.25% w/v orange tint.

### Example 18

A tinted aqueous 2.0% w/v CHG solution using an anionic dye and cationic excipient was prepared using a Class A 100-ml volumetric flask. In this example, the anionic dye used was 0.25% w/v orange tint, which consisted of 75% D&C Yellow No. 8 and 25% FD&C Red No. 40. First, 0.440 grams CPC and 0.25 grams orange tint, consisting of 0.1875 grams of D&C Yellow No. 8 and 0.0625 grams of FD&C Red No. 40, were dissolved in the flask using 50.0 ml of distilled water. Consecutively, 10.6 grams of 20% w/v CHG solution were added. The solution was then brought up to volume with distilled water. The resulting solution had a molar ratio of cationic excipient to anionic dye of 1.79.

Solutions were also prepared using 4.0% w/v CHG with 0.25% w/v orange tint, and 6.0% w/v CHG with 0.25% w/v orange tint. For these solutions, the resulting molar ratio of cationic excipient to anionic dye was also 1.79.

After preparation of the solutions, the 2.0%, 4.0%, and 6.0% w/v CHG solutions had a clear intense orange appearance. The solutions were stored for 24 hours at room temperature. During this period, no visible precipitate formed in any of the solutions, indicating that the solutions were stable and the dyes were compatible for at least 24 hours at a concentration of 0.25% w/v orange tint.

### Example 19

A tinted aqueous 2.0% w/v CHG solution using an anionic dye and cationic excipient was prepared using a Class A 100-ml volumetric flask. In this example, the anionic dye used was 0.50% w/v D&C Yellow No. 8. First, 1.097 grams CPC and 0.50 grams D&C Yellow No. 8 were dissolved in the flask using 50 ml of distilled water. Consecutively, 10.6 grams of 20% w/v CHG solution were added. The solution was then brought up to volume with distilled water. The resulting solution had a molar ratio of cationic excipient to anionic dye of 1.91.

After preparation of the solution was stored for 24 hours at room temperature. During this period, no visible precipitate formed indicating that the solution was stable and the dye was compatible for at least twenty four hours at a concentration of 0.50% w/v D&C Yellow No. 8.

As can be understood, the present invention provides an aqueous antiseptic solution comprising an aqueous solution of cationic antiseptic or a salt thereof and a compatible dye in an amount sufficient to stain a patient's skin.

The present invention has been described in relation to particular embodiments, which are intended in all respects to be illustrative rather than restrictive. Since many possible embodiments may be made of the invention without departing from the scope thereof, it is to be understood that all matter herein set forth or shown in the accompanying drawings is to be interpreted as illustrative and not in a limiting sense. Alternative embodiments will become apparent to those of ordinary skill in the art to which the present invention pertains without departing from its scope. For example, although embodiments of the present invention have been described with respect to disinfecting and coloring a patient's skin, in further embodiments, the aqueous antiseptic solution may be used to disinfect and color other materials and surfaces, such as medical equipment, for example.

From the foregoing, it will be seen that this invention is one well adapted to attain all the ends and objects set forth above, together with other advantages which are obvious and inherent to the system and method. It will be understood that certain features and subcombinations are of utility and may be employed without reference to other features and subcombinations. This is contemplated and within the scope of the claims.

What is claimed is:

1. An antiseptic solution comprising:
a micellular complex consisting of a cationic excipient and an anionic dye; and
a cationic antiseptic.

2. The antiseptic solution of claim 1, wherein said micellular complex consists of stoichiometric amounts of said excipient and said dye.

3. The antiseptic solution of claim 1, wherein said cationic excipient is cetylpyridinium chloride.

4. The antiseptic solution of claim 1, wherein said cationic antiseptic is cetylpyridinium chloride.

5. The antiseptic solution of claim 1, wherein said cationic excipient and said cationic antiseptic comprise different compounds.

6. The antiseptic solution of claim 1, wherein said cationic antiseptic is selected from olanexidine, alexidine, octenidine, hexitidine, hexamidine, polyhexamethylene biguanide, chlorhexidine, and salts thereof.

7. The antiseptic solution of claim 1, wherein said solution is substantially clear.

8. A method of making an antiseptic solution, said method comprising:
forming a micellular complex consisting of a cationic excipient and an anionic dye; and
combining said complex with a cationic antiseptic.

9. The method of claim 8, wherein said micellular complex consists of stoichiometric amounts of said excipient and said dye.

10. The method of claim 8, wherein said cationic excipient is cetylpyridinium chloride.

11. The method of claim 8, wherein said cationic antiseptic is cetylpyridinium chloride.

12. The method of claim 8, wherein said cationic excipient and said cationic antiseptic comprise different compounds.

13. The method of claim 8, wherein said cationic antiseptic is octenidine.

14. The method of claim 8, wherein said solution is substantially clear.

15. An aqueous antiseptic solution comprising an aqueous solution of a cationic antiseptic or a salt thereof, and a cationic dye in an amount sufficient to stain a patient's skin when applied.

16. The aqueous antiseptic solution of claim 15, wherein the cationic antiseptic or the salt thereof comprises at least one of olanexidine, alexidine, octenidine, hexitidine, hexamidine, polyhexamethylene biguanide, chlorhexidine, and salts thereof.

17. The aqueous antiseptic solution of claim 16, wherein the concentration of the octenidine or the salt thereof is from 0.1% w/v to 0.5% w/v.

18. The aqueous antiseptic solution of claim 15, wherein the cationic dye comprises at least one of crystal violet, acriflavine, bismark brown, malachite green, methyl green, Victoria pure blue BO, and azure C.

19. The aqueous antiseptic solution of claim 15, wherein the concentration of the cationic dye is from 0.004% w/v to 0.5% w/v.

20. The aqueous antiseptic solution of claim 15, wherein the aqueous antiseptic solution further comprises a surfactant.
